(19) United States
(12) Patent Application Publication
(10) Pub. No.: US 2007/0173706 A1
(43) Pub. Date: Jul. 26, 2007

(54) METHOD AND APPARATUS FOR INSERTION OF A SENSOR

(75) Inventors: Mark Neinast, Lake Oswego, OR (US); Robert Bruce, Portland, OR (US); W. Kenneth Ward, Portland, OR (US); Richard G. Sass, Portland, OR (US); Jon Fortuna, Mechanicsburg, PA (US)

Correspondence Address: SCHWABE, WILLIAMSON & WYATT, P.C.;PACWEST CENTER, SUITE 1900, 1211 SW FIFTH AVENUE, PORTLAND, OR, 97204, US

(73) Assignee: ISENSE CORPORATION, Portland, OR (US)

(21) Appl. No.: 11/558,394

(22) Filed: Nov. 9, 2006

Related U.S. Application Data
(60) Provisional application No. 60/735,732, filed on Nov. 11, 2005.

(51) Int. Cl. A61B 5/00; A61B 5/05

(52) U.S. Cl. 600/309; 600/345

(57) ABSTRACT

A device and method for delivering a device such as a sensor or fluid transport structure or a fluid transport structure sensor combination into, for example, mammalian skin. Such a device allows a sensor to penetrate mammalian skin without the use of an introducer device such as a needle. A device in accordance with embodiments of the present invention includes a housing for attachment to mammalian skin including an exit port for receiving the distal end of a biosensor and an injection activation device including a mechanism for forcing the sensing device from a first position within the housing, through the exit port to a second position, with sufficiently high velocity to partially penetrate the mammalian skin.

### CROSS REFERENCE TO RELATED APPLICATIONS

The present application claims priority to U.S. Provisional Patent Application No. 60/735,732, filed Nov. 11, 2005, entitled “Method and Apparatus for Insertion of a Sensor” the entire disclosure of which is hereby incorporated by reference in its entirety.

### TECHNICAL FIELD

This present invention relates generally to devices for delivering mechanically slender devices through skin into a body to perform various medical or physiological functions. More specifically the present invention relates to a method for transcutaneous placement of a soft cannula biosensor or flexible biosensor safely and automatically, without the aid of a rigid and or sharp introducer device or the resultant need for disposal of a contaminated sharp introducer device.

### BACKGROUND

There are several instances of medically useful devices which are mechanically slender and flexible and are also inserted through the skin.

For example, sensors facilitate the sensing of certain conditions within a patient. Electrochemical sensors are commonly used to monitor blood glucose levels in the management of diabetes. In one scheme, an electrochemical sensor incorporating an enzyme is fabricated onto a small diameter wire. A second reference electrode is also fabricated around the wire near the sensing electrode. The sensor assembly is inserted through the skin so that it is surrounded by interstitial fluid. A portion of the sensor assembly exits the skin, remaining outside the body, where electrical connections to the sensing electrode and reference electrode may be made. A suitable electronic measuring device outside the body may be used to measure electrical current from the sensor for recording and display of a glucose value. These types of devices are described, for example, in U.S. Pat. No. 5,965,380 to Heller et al. and U.S. Pat. No. 5,165,407 to Ward et al.

In addition to electrochemical glucose sensors, a number of other electrochemical sensors have been developed to measure the chemistry of blood or other body fluids or materials. Electrochemical sensors generally make use of one or more electrochemical processes and electrical signals to measure a parameter. Other types of sensors include those which use optical techniques to perform a measurement.

In other applications, a cannula and sensor combination device is inserted through the skin to allow insulin to be introduced into the body as part of an artificial pancreas system. In these applications, a slender (small cross-section) and flexible device offers several advantages over a larger and more rigid device. Patient comfort is increased, especially during long-term insertion, and trauma at the entry site is reduced. A flexible device also is able to adjust to movement of the skin during physical activity, increasing patient comfort. In many cases these devices will remain inserted in the body for 5 to 7 days.

Although the slender and flexible nature of these devices increases patient comfort, these devices are difficult to insert through the skin. Unlike a typical hypodermic needle, these devices are too fragile and flexible to be simply pushed through the skin surface using normal force and speed. When the tip of such a device is forced against the skin, the device will bend and collapse with much less force than would be required to achieve skin penetration. Although in some cases the tip of the device may be sharpened to ease penetration, this approach is not typically adequate to assure penetration, and some devices such as tubing-based devices are not appropriate for sharpening. Also, the sharpening process adds to production cost and complexity.

As will be understood by those skilled in the art, human skin possesses biomechanical properties influenced by a relatively impenetrable outer layer, the stratum corneum, and inner layers which are more easily penetrated. These biomechanical properties cause penetration of the skin surface to present the primary challenge in introducing a relatively fragile slender, flexible device into the skin.

Current art provides several approaches for insertion of such slender flexible devices through the skin. In one case, the device is placed coaxially inside a hollow tube with a sharpened end, such as a hypodermic needle or trocar. The needle is inserted through the skin with the device inside. As a second step, the needle is withdrawn, leaving the device behind, passing through the skin into the body. See, for example, U.S. Pat. No. 6,695,860 to Ward et al. The insertion process may be painful, due to the large diameter needle, and a larger opening is made in the skin than required for passing the device alone, increasing trauma and the possibility of infection.

In a variation of this approach, the functions of the device are incorporated into a thin needle which must stay inserted into the skin. The needle provides additional mechanical strength and a sharpened point to assist in piercing the skin. However, due to its larger size and rigidity, this approach also contributes to patient discomfort for the duration of the insertion. See, for example, U.S. Pat. No. 6,501,976.

In addition, the presence of a rigid needle places mechanical constraints on the size and shape of the device housing that is attached to the surface of the skin where the device exits the skin. The needle also must be treated as a biohazard “sharp” since it is capable of transmitting disease if it should accidentally puncture the skin of another individual after being used in device insertion.

### BRIEF DESCRIPTION OF THE DRAWINGS

Embodiments of the present invention will be readily understood by the following detailed description in conjunction with the accompanying drawings. To facilitate this description, like reference numerals designate like structural elements. Embodiments of the invention are illustrated by way of example and not by way of limitation in the figures of the accompanying drawings.

FIG. 1 illustrates a block diagram of an insertion device according to an embodiment of the present invention;

FIG. 2A illustrates an embodiment of an electrochemical glucose sensor that has been fabricated onto a length of thin, flexible wire in accordance with embodiments of the present invention;

FIG. 2B shows a cross-section of how an electrochemical sensor may appear when inserted into skin in accordance with an embodiment of the present invention;

FIG. 3A shows an insertion device according to embodiments of the invention in which a plunger and spring combination is utilized to insert an electrochemical sensor;

FIG. 3B shows an insertion device according to embodiments of the invention in which a sensor may be initially retracted from the skin and initially in contact with a plunger;

FIG. 4 shows an embodiment of the invention with a reduced guide and support structure;

FIG. 5A shows an embodiment of the invention in which the insertion device includes a transmitter top and a sensor base;

FIG. 5B shows an embodiment of the invention prior to the attachment of a transmitter top and a sensor base;

FIG. 6A shows an embodiment of the invention in which the components of a sensor base are exposed to view;

FIG. 6B shows an embodiment of the invention in which only some of the components of a sensor base are exposed to view’

FIG. 6C shows a cross sectional view of a sensor base in accordance with an embodiment of the invention;

FIG. 7A shows a guidance concept in accordance with an embodiment of the present invention in which a sensor is guided using three plastic guides;

FIG. 7B shows a guidance concept in accordance with an embodiment of the present invention in which the sensor has attached two metallic guides that may double as conductors;

FIG. 7C shows a guidance concept in which spring contacts may be mated to metallic guides that may double as conductors;

FIG. 8 shows an embodiment of the invention in which energy stored in a curved sensor is utilized to provide motive force to the sensor;

FIG. 9A shows an embodiment of the invention in which a linear solenoid is utilized to provide motive force to a sensor;

FIG. 9B shows an embodiment of the invention in which a rotary solenoid is utilized to provide motive force to a sensor;

FIG. 10 shows an embodiment of the invention in which a CO₂ cartridge is utilized to provide motive force to a sensor;

FIG. 11 shows an embodiment of the invention in which an air pump and piston are utilized to provide a motive force to a sensor;

FIG. 12 shows an embodiment of the invention in which a mechanical spring is utilized to provide a motive force to a sensor and the activation is controlled by a separate bowed spring;

FIG. 13A shows an embodiment of the invention in which a mechanical spring and slider combination is utilized to provide a motive force to a sensor;

FIG. 13B shows a cross sectional view of an embodiment of the invention in which a mechanical spring and slider combination is utilized to provide a motive force to a sensor;

FIG. 14 shows an embodiment of the invention in which a series of mechanical springs and a shear member are used to control and provide a motive force to a sensor;

FIG. 15 shows an embodiment of the invention in which electrical connection is made to a sensor via wires insert molded and soldered onto the conductive regions of the sensor;

FIG. 16A shows an exploded view of an embodiment of the invention that utilizes a canted coil spring probe termination to make electrical contact to the sensor;

FIG. 16B depicts an assembled view of an embodiment of the invention that utilizes a canted coil spring probe termination to make electrical contact to the sensor;

FIG. 17A shows an embodiment of the invention in which a paper guidance structure is utilized both to secure a sensor prior to insertion and to guide the sensor during insertion; and

FIG. 17B shows a view of an embodiment of the invention after sensor insertion in which a paper guidance structure has been utilized to guide the sensor during insertion.

### DETAILED DESCRIPTION OF THE EMBODIMENTS OF THE INVENTION

In the following detailed description, reference is made to the accompanying drawings which form a part hereof wherein like numerals designate like parts throughout, and in which is shown by way of illustration embodiments in which the invention may be practiced. It is to be understood that other embodiments may be utilized and structural or logical changes may be made without departing from the scope of the present invention. Therefore, the following detailed description is not to be taken in a limiting sense, and the scope of embodiments in accordance with the present invention is defined by the appended claims and their equivalents.

Various operations may be described as multiple discrete operations in turn, in a manner that may be helpful in understanding embodiments of the present invention; however, the order of description should not be construed to imply that these operations are order dependent.

The description (including the claims) may use perspective-based descriptions such as up/down, back/front, and top/bottom. Such descriptions are merely used to facilitate the discussion and are not intended to restrict the application of embodiments of the present invention.

For the purposes of the present invention, a phrase in the form “A/B” means A or B. For the purposes of the present invention, a phrase in the form “A and/or B” means “(A), (B), or (A and B)”. For the purposes of the present invention, a phrase in the form “at least one of A, B, and C” means “(A), (B), (C), (A and B), (A and C), (B and C), or (A, B and C)”. For the purposes of the present invention, a phrase in the form “(A)B” means “(B) or (AB)” that is, A is an optional element.

The description may use the phrases “in an embodiment,” or “in embodiments,” which may each refer to one or more of the same or different embodiments. Furthermore, the terms “comprising,” “including,” “having,” and the like, as used with respect to embodiments of the present invention, are synonymous.

For the purposes of describing embodiments of the present invention and the claims that follow, the term “high speed motive force” refers to a force sufficient to drive a thin, flexible medical device into animal skin—including the relatively impenetrable outer layer, the stratum corneum, as well as the inner layers which are more easily penetrated—without substantial bending or substantial deflection of the sensor, such as a force of approximately 0.5 N/mm to 10 N/mm. As would be obvious to one of ordinary skill in the art, the force necessary to drive a thin, flexible medical device into animal skin increases if the medical device encounters resistance other than that provided by the surface of animal skin such as, for example, scar tissue or frictional resistance caused by a guidance structure or tube that the medical device must pass through. The term “high speed motive force” encompasses force necessary to drive the thin, flexible medical device into animal skin in situations where the medical device may encounter such other resistance. Stated another way, the term “high speed motive force” encompasses any amount of motive force necessary to be applied to a thin, flexible medical device such that the sum of all forces acting on the medical device as the motive force is applied is sufficient to drive it into animal skin.

The term “actuator” refers to any of various electric, hydraulic, magnetic, pneumatic, or other means by which something is moved or controlled. The term “solenoid actuator” refers to a variety of electromechanical devices that convert electrical energy into linear or rotational motion. The term “trigger” indicates any of various electric, hydraulic, magnetic, pneumatic, or other means of initiating a process or reaction. The term “sabot” indicates a thick circular disk with a center hole.

For the purposes of describing embodiments of the present invention and in the claims that follow, the term “axial support” means the support or bracing of a relatively straight, slender object when a motive force is applied to the object in such a way as to resist force vectors acting perpendicular to an imaginary line drawn through the device lengthwise; such support or bracing sufficient to prevent or reduce crimping, creasing, folding, or bending of the straight, slender object; or such support or bracing sufficient to enable the object to return to a relatively straight configuration after minimal bending such that the object substantially retains its original shape with minimal crimping, creasing, folding, or bending.

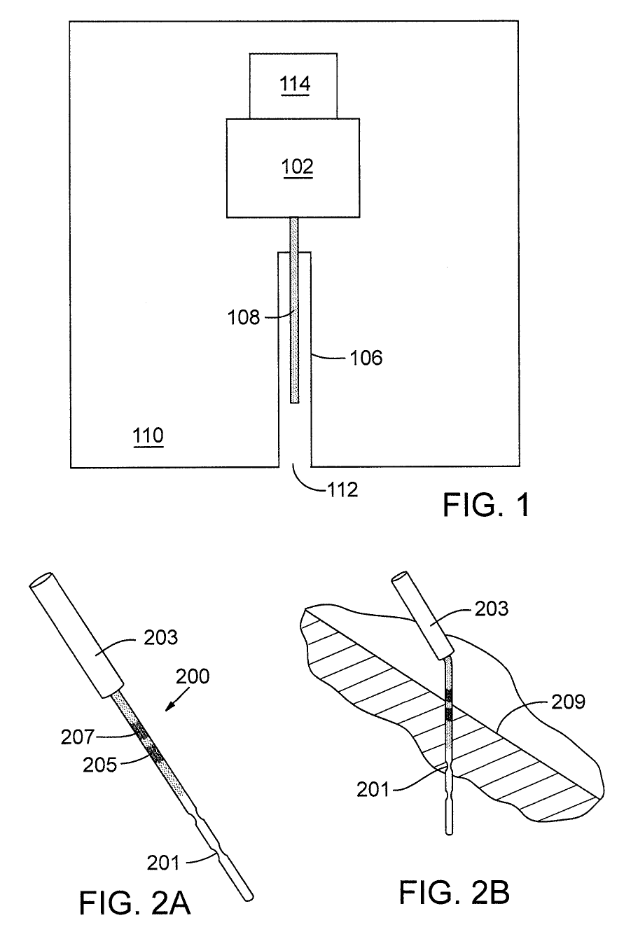

For the purposes of describing embodiments of the present invention and in the claims that follow, the term “associated with” indicates that an object, element, or feature is coupled to, connected to, or in proximity to and in communication with another object, element, or feature. For example, as depicted in FIG. 1, mechanism 102 may apply a high speed motive force to analyte sensor 108 such that analyte sensor 108 moves through guidance structure 106. Mechanism 102 is therefore both proximally near guidance structure 106 and in communication with guidance structure 106 and is thus “associated with” guidance structure 106.

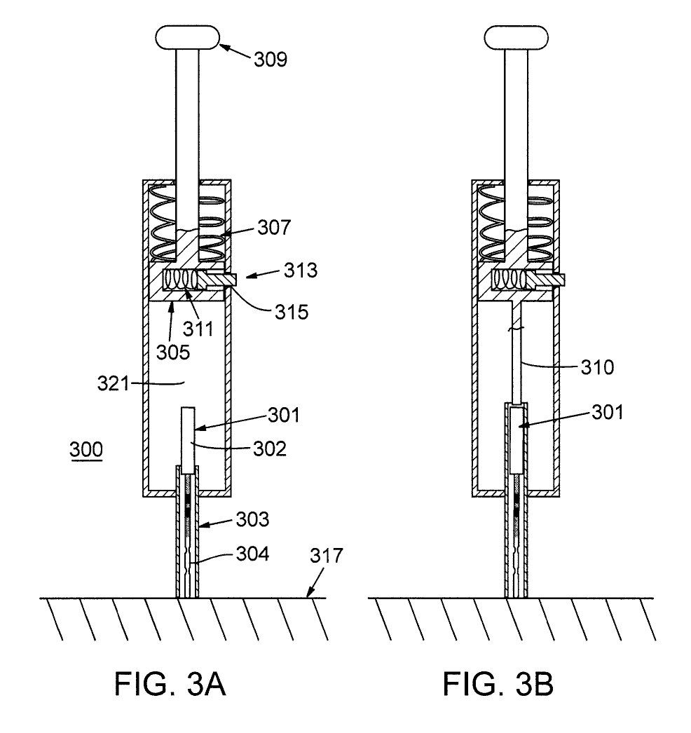

In another example, shown in FIG. 3A, spring 307 may force plunger 305 down toward sensor 301 and may drive sensor 301 through guidance structure 303. Therefore, plunger 305 and spring 307 are in communication with guidance structure 303 and are thus “associated with” guidance structure 303. Plunger 305 and spring 307 may or may not make physical contact with guidance structure 303, and may or may not be in contact when in a static position. Also in FIG. 3, spring 307 is associated with plunger 305 in that spring 307 is connected to plunger 305.

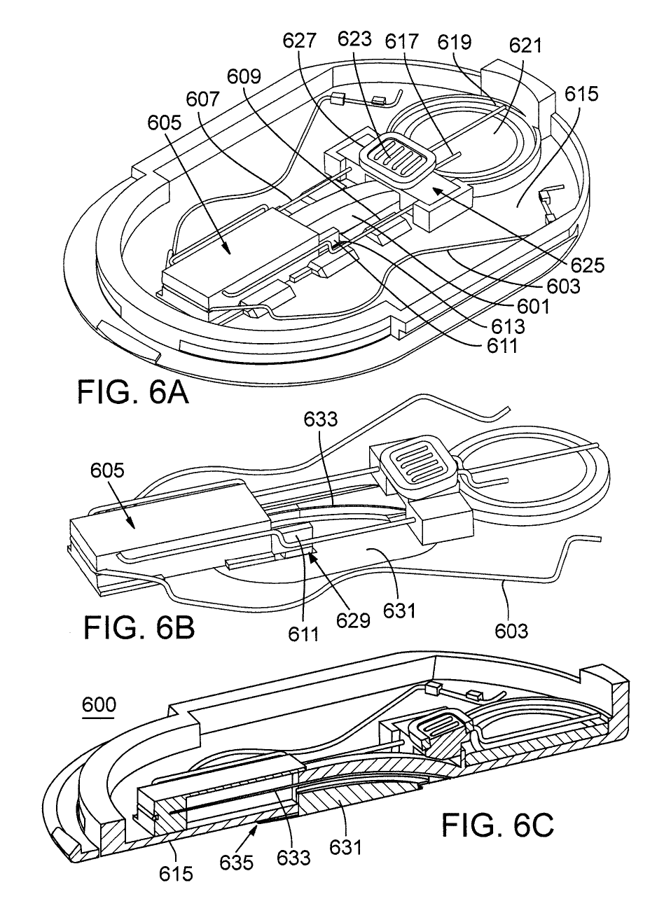

In another example, shown in FIG. 6A, slider 605 is coupled to guidance structure 601 and insertion spring 603 may force slider 605 to move over the top of guidance structure 601. In such a way, both insertion spring 603 and slider 605 are “associated with” curved guidance structure 601.

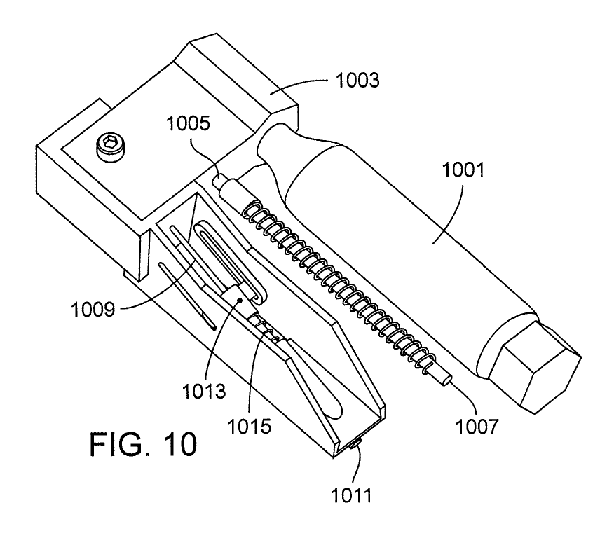

In yet another example shown in FIG. 10, CO₂ cartridge 1001 may release CO₂ gas into manifold 1003 which may allow the gas to pass through an internal valve (not shown) and enter hollow pin 1009 which may force rod 1011 forward striking a sensor (not shown) for insertion. Therefore CO₂ cartridge 1001 is in communication with a sensor (not shown) and thus “associated with” the sensor.

For the purposes of describing embodiments of the present invention and in the claims that follow, the term “guide member” means a device that at least partially axially surrounds the analyte sensor and is adapted to fit inside the guidance structure such that the guide member at least partially occupies at least some part of the space between the sensor and the guidance structure either during insertion, before insertion, and/or after insertion. A guide member may either provide axial support; assist a sensor in moving through the guidance structure; or both. Exemplary guide members include a sabot, a spiral of plastic, a rectangular metallic guide, an open cell foam plastic cylinder, and a thin plastic disk. As will be appreciated by one of ordinary skill in the art, a guide member may be made of many different materials and shaped in various geometries which may or may not correspond to the geometry of the guidance structure.

For the purposes of describing embodiments of the present invention and in the claims that follow, the term “electrical network” means electronic circuitry and components in any desired structural relationship adapted to, in part, receive an electrical signal from an associated sensor and, optionally, to transmit a further signal, for example to an external electronic monitoring unit that is responsive to the sensor signal. The circuitry and other components may or may not include a printed circuit board, a tethered or wired system, etc. Signal transmission may occur over the air with electromagnetic waves, such as RF communication, or data may be read using inductive coupling. In other embodiments, transmission may be over a wire or via another direct connection.

An embodiment of the present invention may include, as shown in FIG. 1, a mechanism 102 adapted to generate a high speed motive force coupled to a guidance structure 106 which may be adapted for insertion of an analyte sensor 108. Mechanism 102 may be controlled by trigger 114. In various embodiments of the invention, analyte sensor 108 may be driven by a high speed motive force generated by mechanism 102 through the guidance structure and out of guidance structure opening 112. In FIG. 1, guidance structure opening 112 is shown flush with the edge of housing 110. However, in embodiments, the guidance structure opening may be placed either outside of housing 110 or nested inside a larger opening of housing 110.

In embodiments, a guidance structure may be a hollow tube with a circular cross-section. In embodiments, a guidance structure may be linear. In embodiments, a guidance structure may be curved to allow motive force to be applied to a sensor in a direction other than perpendicular to the skin in which the sensor is to be inserted. In embodiments, a guidance structure may be a curved hollow tube with a circular cross-section.

In various embodiments, the edge of housing 110 where opening 112 is situated may be placed flush against skin prior to insertion. Placing the edge of housing 110 flush against the skin may generate tension on the skin surface which may assist in inserting the sensor without buckling or deflection of the sensor. In an embodiment in which guidance structure 112 extends beyond the surface of housing 110, it may be the pressure of guidance structure 112 against that skin that may provide tension to the skin.

FIG. 2A shows an analyte sensor 200 that may be inserted according to various embodiments of the present invention. In FIG. 2A, analyte sensor 200 is an electrochemical glucose sensor that has been fabricated onto a length of thin, flexible wire. A reference or ground electrode 205 and a sensing electrode 207 may be incorporated into analyte sensor 200. Small diameter end 201 (proximal end) of sensor 200 may be inserted through the skin. In an embodiment, this diameter may be approximately 0.25 mm or less. In an embodiment, on the larger diameter end (distal end) of sensor 200, its diameter has been increased by adding a sleeve of steel tubing 203 which may increase its rigidity and facilitate electrical connections. In some embodiments, the diameter of the larger section may be, for example, approximately 0.5 mm. In an embodiment, the larger diameter portion of the sensor may remain outside of the body upon insertion. FIG. 2B shows a cross-section of the sensor when inserted into the skin. In some embodiments, a 10-20 mm, for example approximately 15 mm, length of sensor 200 may be implanted beneath the skin.

In embodiments, a sensor inserted according to an embodiment of the present invention may be rigid or flexible. In some embodiments, a flexible sensor is one that may be flexed repeatedly, such as the type of flexion experienced by a subcutaneously implanted sensor in a human during normal movement, over a period of time (such as 3-7 days or more) without fracture. In an embodiment, a flexible sensor may be flexed hundreds or thousands of times without fracture.

FIG. 3A shows an insertion device in accordance with an embodiment of the present invention. Sensor 301 may be placed into guidance structure 303 within insertion device 300. In an embodiment, guidance structure 303 may allow free passage of larger diameter end 302 of sensor 301 while providing axial support. Guidance structure 303 may also provide some axial support to the smaller diameter end 304 of sensor 301, although there may be more clearance between the inside of guidance structure 303 and sensor 301 at small diameter end 304. In an embodiment, guidance structure 303 may provide axial support to the sensor in order to successfully drive sensor 301 into the skin.

Insertion device 300 may also contain plunger 305, compression spring 307 and a release mechanism consisting of spring 311 and pin 313. In preparation for sensor insertion, plunger 305 may be withdrawn against spring 307 using handle 309 creating tension in spring 307. The release mechanism holds plunger 305 in position. To implant sensor 301, pin 313 may be forced into the body of plunger 305 through slot 315, thus compressing spring 311 and freeing plunger 305 and allowing spring 307 to force plunger 305 down barrel 321 of insertion device 300 to strike large diameter end 302 of sensor 301. Plunger 305 may drive sensor 301 into position in skin 317. Upon insertion, insertion device 300 may be withdrawn over the end of sensor 301 without disturbing its location in skin 317.

In an embodiment, appropriate electrical connections may be made after insertion device 300 is withdrawn. In an alternative embodiment, insertion device 300 may be integrated with a sensing device or an associated housing that has various electrical components, including electrical connections to sensor 301. In such an embodiment, the electrical components may be connected to sensor 301 prior to insertion, and upon insertion, insertion device 300 may be withdrawn by manipulation through a slot present in guidance structure 303 and/or in insertion device 300. In other words, guidance structure 303 and/or insertion device 300 may be configured with a slot (straight or curved) to allow removal of either device from association with sensor 301 even while sensor 301 is electrically connected at its distal end (large diameter end) to additional electrical components.

It will be appreciated by those skilled in the art that numerous alternatives are possible for the guide and support structures, spring, plunger and release mechanism which fulfill the various purposes of embodiments of the present invention for supporting the sensor and for providing a controlled impact and driving force.

It will also be appreciated that while a wire-based electrochemical glucose sensor may be used, similarly-shaped devices, such as other sensors or drug delivery devices such as small tubing used to dispense insulin or another medication may be substituted for the glucose sensor in embodiments of the present invention.

In an embodiment, an insertion mechanism may be used only once as part of a disposable assembly. In such an embodiment, there may be no need to provide a manual means to withdraw the plunger and set the release mechanism by the user, as the device may be assembled with the plunger already withdrawn and the release mechanism set and ready for insertion.

To puncture the skin without damaging the sensor, a high initial impact of the sensor tip against the skin may be utilized followed by a controlled driving force to complete the insertion through the softer inner skin layers. Note that an embodiment of the insertion device shown in FIG. 3A provides for a space or distance between the withdrawn plunger and the end of the sensor that will be driven.

In embodiments such as shown in FIG. 3A, the force of the spring may cause the plunger to accelerate through this distance before striking the end of the sensor. The velocity of the plunger provides additional initial impact to the sensor that may assist in driving it through the tough outer layer of skin quickly. In an embodiment, the force of the spring alone may be sufficient to complete the insertion.

In other embodiments, the high initial impact of the sensor tip against the skin may be achieved in other ways. For example, in another embodiment, shown in FIG. 3B, sensor 301 may be initially retracted from the skin and may be initially in contact with plunger 310. In this embodiment, sensor 301 may be accelerated along with plunger 310 before impacting the skin.

In yet other embodiments, the sensor alone may be accelerated by a motive force to achieve momentum causing an impact sufficient to penetrate the skin.

It will be understood by one of ordinary skill in the art that in other embodiments of the invention, means other than a spring may be utilized to provide a high speed motive force. Some examples include an electric solenoid, a shape memory alloy spring which provides an electrically initiated driving force, an associated CO₂ cartridge, a compressed air pump, etc.

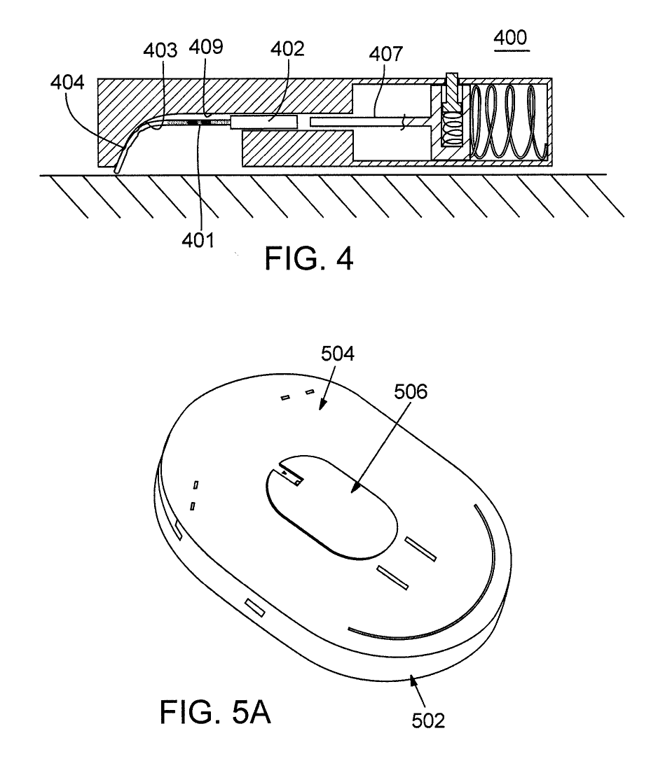

FIG. 4 shows an embodiment of insertion device 400 with a reduced and curved guide and support means. In an embodiment, prior to insertion, sensor 401 is supported at its larger end 402. Thin distal end 404 of sensor 401 follows a curved path during insertion. However, in this case, guidance structure 409 may consist primarily of a partially open region with a curved section 403 which may guide and support the sensor on only one side of sensor 401 that lies outside the radius of the arc formed by sensor 401 during insertion. It will be understood by those skilled in the art that while insertion force is applied, sensor 401 may exert a radial outward force against the supporting wall of guidance structure 409 of insertion device 400 along curved section 403. This radial force may tend to support and stabilize sensor 401 without the need for a completely surrounding guidance structure.

Another feature of the embodiment in FIG. 4 is that the open region at the skin contact side of guidance structure 409 may allow the sensor to be easily and completely freed from insertion device 400 when insertion is complete. In addition, in an embodiment, the open region may be large enough that additional electrical connections and/or components associated with sensor 401 may be accommodated before, during, and/or after insertion.

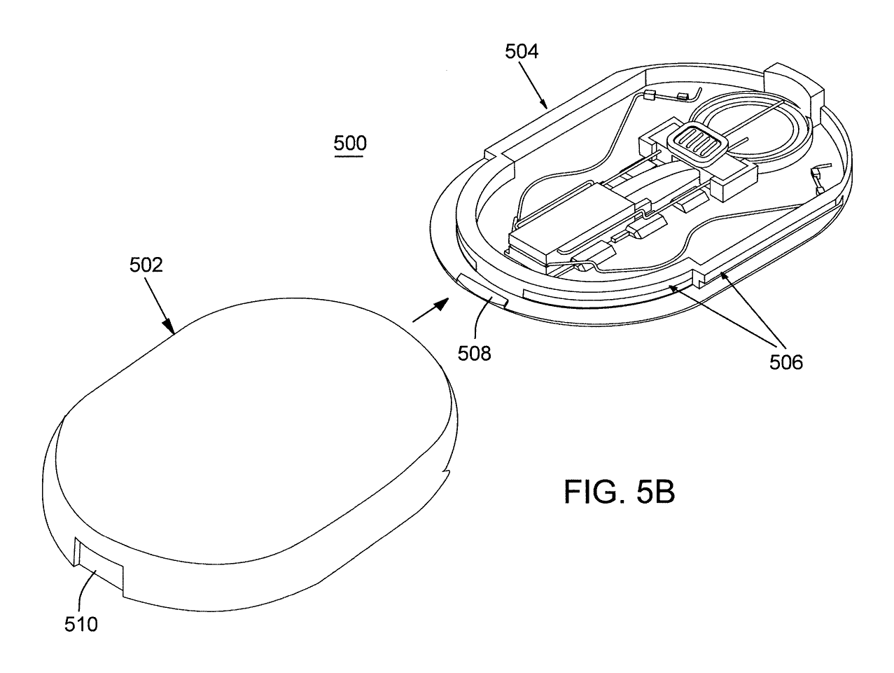

FIG. 5A depicts an embodiment of the invention wherein the assembled insertion device may include a transmitter 502, a sensor base 504, which may, in an embodiment, be disposable, and a probe trigger 506. In this embodiment, a sensor and a means for supplying a high speed motive force to the sensor (not shown) may be contained within sensor base 504. In an embodiment, the sensor may be inserted by placing the bottom of the sensor base 504 onto the skin and pressing on the top of transmitter 502 (in a press fit, snap fit, or other type of arrangement) causing probe trigger 506 to move or otherwise be triggered causing the means for supplying a high speed motive force inside sensor base 504 to strike the sensor thereby inserting it into the skin.

The embodiment depicted in FIG. 5A may include disposable or reusable portions such as sensor base 504 and transmitter 502. Thus, in an embodiment, a resposable device may be provided comprising a reusable transmitter component 502 and a disposable sensor base 504. In embodiments, other electrical components (battery, processing components, etc.) may be provided in either transmitter component 502 and/or sensor base 504.

The transmitter component may contain circuitry in accordance with an embodiment of the present invention which may include an electrical network adapted to receive an electrical signal from an associated sensor and to transmit a further signal, for example to an external electronic monitoring unit that is responsive to the sensor signal. In embodiments, an electrical network may comprise a variety of components in any desired structural relationship, whether or not the network has a printed circuit board, a tethered or wired system, etc. In an embodiment, signal transmission may occur over the air with electromagnetic waves, such as RF communication, or data may be read using inductive coupling. In other embodiments, transmission may be over a wire or via another direct connection.

In an embodiment of the invention, shown disassembled in FIG. 5B, sensing device 500 may be assembled by sliding transmitter 502 into grooves 506 on sensor base 504. Grooves 506 on sensor base 504 align and secure sensor base 504 and transmitter 502 together. In an embodiment, locking latch 508 secures to locking edge 510 to provide additional securing.

In an embodiment, a transmitter may be reused while the sensor base may be adapted to be used once and discarded. In other embodiments, the sensor base and transmitter may both be reused. In still other embodiments, both may be adapted to be discarded.

In embodiments of the present invention, a handtool may be used to assemble the transmitter and sensor base together. The handtool may be used by first placing the transmitter upside down on the handtool. The sensor base may be provided with tape strip and a backing card situated along the bottom of the sensor base in place and with a protective bubble cap over the opposite face. The bubble cap may be removed from the sensor base and the sensor base may then be placed on to a sliding member of the handtool. The backing card may be used to align the sensor within the handtool. Next, the sliding member may be pushed over the transmitter snapping the transmitter and sensor base together. In an alternative embodiment, the handtool may have two components that hinge together rather than a sliding member. After assembly, the backing card may be removed and the tool may be used to position the device on a patient's body. In embodiments, by pushing on the tool, the trigger may move, activating an injection activation device and the sensor may be inserted in the patient. The handtool may be released by squeezing on release tabs. It will be apparent to one of ordinary skill in the art that many different embodiments of a handtool could be utilized, or, in embodiments, no handtool may be used.

In some embodiments, the means for supplying a high speed motive force may be attached to the sensor base. In other embodiments, the means for supplying a high speed motive force may be attached to the transmitter. In embodiments, the means for supplying a high speed motive force may be in a separate handle not part of either the sensor base or the transmitter. In embodiments, such a handle may be removed after insertion. Details about such a handle may be found in U.S. patent application Ser. No. 11/468,673, which describes a device that uses a handle to provide motive force to insert a sensor also employing a trocar. Although the present invention primarily involves a method and apparatus to insert a sensor without using a trocar or related device, details from U.S. patent application Ser. No. 11/468,673—including the handle—may be extended to various embodiments of the present invention.

FIG. 6A shows components of sensor base 600 in accordance with an embodiment of the invention. Curved guidance structure 601 may be coupled to insertion spring 603 via slider 605 which may house the upper end of a curved probe (not shown). Leads 607 and 609 may be soldered to the sensor to make electrical contact. Thus, slider 605 may provide a housing for insert-molding thereby sealing the terminations and providing protection for the otherwise exposed probe.

Insertion spring 603 may be attached during manufacturing and pulled back over the outermost end of slider 605. Slider 605 may be kept from moving forward by two beams 611 (only one shown) which protrude from slider 605 and engage the edges of rectangular holes 613 in base surface 615 of sensor base 600. In this manner, insertion spring 603 holds potential energy and slider 605 may remain stationary.

Battery leads 617 and 619 may be, for example, spot welded to battery 621 and battery 621 may be secured in place using a potting compound (not shown) or other suitable securing compound or mechanical means. All four leads 607, 609, 617, and 619 may be attached to small wire springs 623 that may be insert-molded into connector assembly 625. A soft rubber gasket 627 may be attached to the periphery of connector assembly 625 for sealing with a corresponding contact pad on the transmitter (not shown) once the transmitter is secured into place. The connection face of connector assembly 625 is on an angle so that the contacts and sealing features do not interfere during mating and so that the total mating forces do not act to try to disengage the transmitter and sensor base 600.

FIG. 6B shows an exploded view of some components of sensor base 600. In this view, guidance structure 601 is omitted exposing probe 633 and riser 629 of trigger 631. In this embodiment of the invention, riser 629 may be pressed upward which in turn may push the two rectangular beams 611 upward causing them to slide against the forward edges of rectangular holes 613 (see FIG. 6A) and be released. Once released, insertion spring 603 may no longer encounter resistance and may cause slider 605 to quickly move forward. In so doing, curved probe 633 will pass through the curved guidance structure and partially pass through an opening (not shown) in the sensor base and may then be inserted into the skin of a patient.

In this embodiment of the invention, trigger 631 may be activated by placing the apparatus on the skin of a patient and applying downward pressure causing trigger 631 and, thus, riser 629, to rise upward in relation to the device.

FIG. 6C depicts a cross-sectional view of sensor base 600. Here trigger 631 is more clearly shown. A curved feature on the top of trigger 631 may hold probe 633 in place before insertion and may help guide curved probe 633 during insertion. Gap 635 between trigger 631 and base surface 615 may close when trigger 631 is pushed up during insertion.

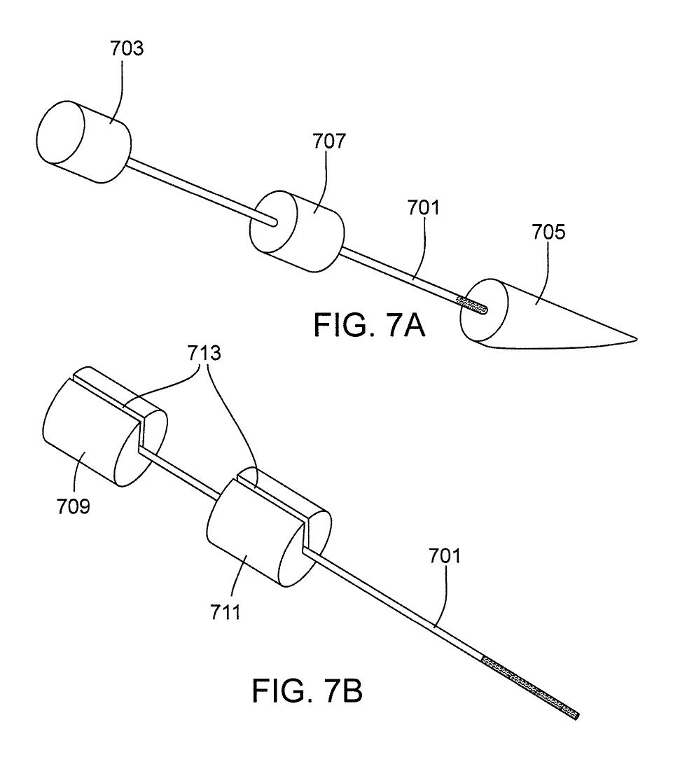

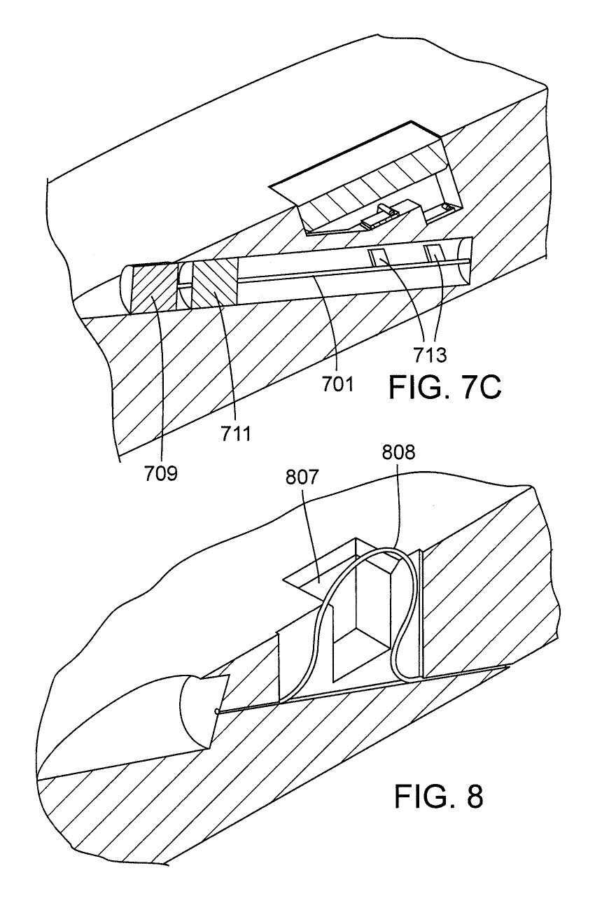

FIG. 7A depicts a probe guidance concept in accordance with an embodiment of the present invention. Sensor 701 is shown with a permanently attached top guide 703. In an embodiment of the invention, top guide 703 may be insert-molded onto sensor 701. In another embodiment, top guide 703 may be attached with adhesive bonding. In other embodiments, top guide 703 may be ultrasonically welded. Lower end guide 705 may be part of the housing body of the device (not shown). Upon insertion, sensor 701 slides within lower end guide 705 which may be a molded feature of the housing body. In another embodiment, lower end guide 705 may be a separate piece bonded to the housing body during manufacturing.

Lower end guide 705 may be angled to allow sensor 701 to be inserted into the skin at an angle other than 90-degrees relative to the skin. In other embodiments of the invention, sensor 701 may be inserted at other angles from 0-90 degrees, including 90 degrees.

Central sabot guide 707 may be free-floating and may remain roughly centrally located on sensor 701 as sensor 701 is inserted into the skin. In other words, in an embodiment of the invention, central sabot guide 707 may be bonded to neither sensor 701 nor the insertion device. Central sabot guide 707 may prevent buckling of sensor 701 upon insertion. All components of FIG. 7 may remain with the device after sensor 701 is inserted.

Although the guidance concept in FIG. 7A is shown with three guides, it will be understood by one of ordinary skill in the art that more than three guides or less than three guides may be employed to guide the sensor and prevent buckling. Although the guidance concept depicted in FIG. 7 is shown with cylindrical guides, it will be understood by one of ordinary skill in the art that other geometries could be employed including, but not limited to, rectangular geometries. In various embodiments, the guides may be shaped and sized to accommodate the shape and size of the guidance structure.

It will be understood by one of ordinary skill in the art that the guides depicted in FIG. 7A may be produced from a variety of materials including, but not limited to, various plastics or metals.

In some embodiments of the invention, the central guide may be composed of open cell foam plastic which may easily collapse during insertion and have virtually no elasticity once compressed.

In another embodiment, the central guide may be a spiral of plastic with a center hole that may serve to guide the probe and prevent buckling during insertion. The spiral may collapse during insertion and take up very little space when compressed. It may remain within the body of the device upon insertion of the sensor. Manufacture of the plastic spiral may be accomplished by molding or by employing a device similar to a rotini pasta extruder.

In another embodiment of the invention, the central guide may be replaced by a series of thin plastic disks each with a central hole. The disks may guide the probe and prevent buckling during insertion. Upon insertion, the disks may close upon each other and take up very little space when compressed. In various embodiments of the invention, the disks may be molded or stamped from a thin sheet of plastic.

In the embodiment of the invention depicted in FIG. 7B, top guide 709 and central guide 711 may facilitate the making of an electrical connection to sensor 701 as well as helping to guide sensor 701 and prevent buckling during insertion. In these embodiments, the guides may be made of a suitable conductive material including any number of suitable metals. In an embodiment, top guide 709 may be soldered to an exposed core of the sensor (not shown) and central guide 711 may be soldered to silver cladding (not shown) via grooves 713. Soldering top guide 709 to sensor 701 may create a permanent attachment to sensor 701 and allow a mechanism for applying a high speed motive force (not shown) to act directly against top guide 709 during insertion.

Referring now to FIG. 7C which shows a cross-sectional view of an embodiment of the sensor and guide design of FIG. 7B placed into an insertion device, electrical contact may be made between the device and guides 709 and 711 by employing a set of leaf spring contacts 713 built into the body of the device. Contact may be made near the end of the travel of sensor 701 upon insertion. In other embodiments, electrical contact may be made by soldered wires that are dressed away from sensor 701 between the top and central guides 709 and 711, respectively.

FIG. 8 depicts a cross-sectional view of the bottom of an insertion device in accordance with an embodiment of the present invention. Sensor 801 is shown bowed and restrained within the body of the device. The top curve of bowed sensor 801 may extend slightly out of exposed opening 807. As depicted in FIG. 8, exposed opening 807 is situated on the bottom surface of the device (the surface adapted to be placed onto the skin). The device may be placed against the skin of a patient (not shown) and pressed down. Force may be applied to the top of bowed sensor 801 to force sensor 801 to straighten forcing proximal tip/end of sensor 801 into contact with the skin with enough pressure to cause sensor 801 to penetrate the skin. Sensor 801 may contain core material with sufficient elastic properties to store a sufficient amount of energy when bowed in order to generate a high speed motive force when straightened.

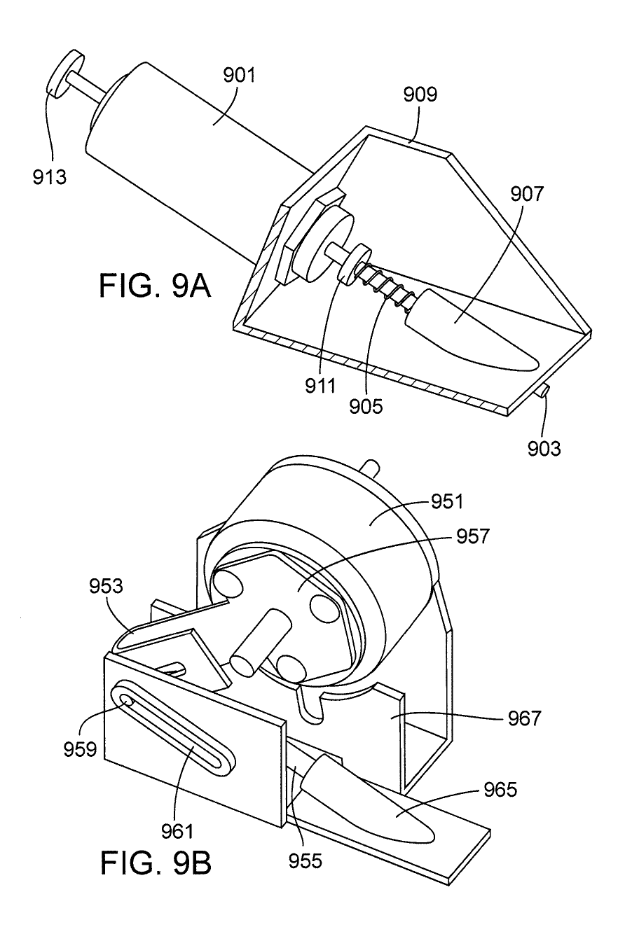

In various embodiments, the direct drive linear solenoid actuator design of FIG. 9A may be employed to provide a high speed motive force to a sensor. In these embodiments, solenoid 901 may be coupled to the main body of the device using support structure 909. Support structure 909 includes cylindrical member 907 which contains a hollow core. Solenoid shaft 903 may be extended so that it also becomes an insertion rod directly impacting and providing a high speed motive force to the end of a sensor (not shown). In an embodiment, solenoid shaft 903 may be partially situated in cylindrical member 907. When power is applied to solenoid 901, shaft 903 may travel through cylindrical member 907 to provide a high speed motive force to a sensor for insertion. After insertion, return spring 905, situated between the end of cylindrical member 907 and shaft stop 911, may cause the shaft to return to its pre-insertion position.

In various embodiments, the rotary solenoid actuator design of FIG. 9B may be employed to provide a high speed motive force to a sensor. In these embodiments, a rotary solenoid 951 may be coupled to the main body of the device using support structure 967. An arm 953 may be attached to the solenoid's rotating plate 957 and the far end of the arm may be slotted and bent back on itself providing an opening for engaging pin 959 attached to the top end of rod 955. Whenever power is applied to solenoid 951, it turns clockwise (as oriented in FIG. 9B) which may cause rotating plate 957 to rotate and pin 959 to move along linear guide slot 961. The linear motion of pin 959 may cause associated rod 955 to move in a linear direction through hollow cylindrical member 965 which is part of the housing structure of the device. Rod 955 may then impact the end of a sensor (not shown) and provide a high speed motive force for insertion of the sensor.

In various embodiments, the rod may return to its original position whenever power is removed from the solenoid. In embodiments, a spring may be incorporated into the solenoid by the manufacturer to ensure that it returns to the rest position whenever power is removed.

It will be appreciated by those of ordinary skill in the art that embodiments of the invention which utilize solenoids are not limited by the configurations depicted in FIGS. 9A and 9B. For example, the rotary solenoid embodiments depicted in FIG. 9B may incorporate a cam surface rather than a rotating arm connected to rotating plate. Embodiments which use a linear solenoid actuator as in FIG. 9A may incorporate intermediate components in various configurations to impact the end of the sensor rather than utilizing an elongated solenoid shaft as depicted in FIG. 9A.

FIG. 10 depicts an embodiment of the invention employing a CO₂ cartridge. As depicted, the head of CO₂ cartridge 1001 may be placed into a hole in manifold 1003 and a nut behind CO₂ cartridge 1001 tightened causing CO₂ cartridge 1001 to move deeper into the manifold where a hollow pin (not shown) pierces CO₂ cartridge 1001 and allows the compressed CO₂ to enter the system. There are two internal manifold chambers (not shown). One chamber connects to CO₂ cartridge 1001 and the other connects to hollow pin 1009. A spring loaded valve (not shown) may be located between them to initially hold back pressure from cartridge 1001 and its associated manifold chamber. Whenever spring loaded firing pin 1007 is allowed to strike valve head 1005, an internal valve (not shown) temporarily opens and an amount of gas may flow from the manifold chamber associated with CO₂ cartridge 1001 into the manifold chamber associated with hollow tube 1009. Gas may then enter hollow tube 1009 and force rod 1011 to move forward and strike a sensor (not shown) for insertion. As rod 1011 nears the end of travel, exhaust port 1013 may travel past the end of hollow tube 1009 allowing the CO₂ to escape. Return spring 1015 may be employed to move rod 1011 back to its original position after insertion.

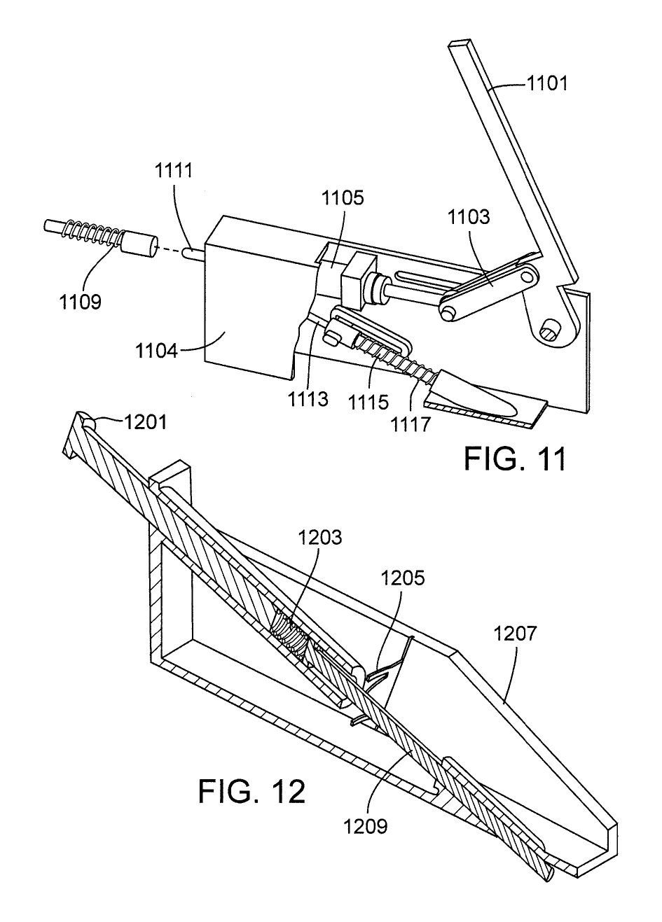

An embodiment of the invention employing an air pump is depicted in FIG. 11 in a cross-sectional view. The embodiment shown in FIG. 11 may employ a similar manifold system as in the CO₂ cartridge embodiment discussed previously. The manifold is encased in housing structure 1104. When lever arm 1101 is pulled up, air may be sucked into a manifold chamber associated with piston 1105 via a one-way valve (not shown). Pushing lever arm 1101 down moves link 1103 which is coupled to the shaft of piston 1105 which may then be forced into its associated manifold. The motion of piston 1105 into the manifold may compress the air that has been sucked into the associated manifold chamber on the upward stroke of lever arm 1101. When spring loaded firing pin 1109 is allowed to strike valve head 1111, an internal valve (not shown) temporarily opens and compressed air may move from the manifold chamber associated with piston 1105 into a manifold chamber associated with hollow tube 1113. Gas may then enter hollow tube 1113 and force rod 1115 to move forward and strike a sensor (not shown) for insertion. As rod 1115 nears the end of travel, an exhaust port on the rod (not shown) may travel past the end of hollow tube 1113 allowing the compressed gas to escape. Return spring 1117 may be employed to move rod 1115 back to its original position after insertion.

FIG. 12 depicts an embodiment in accordance with the present invention employing a mechanical spring. In this embodiment, bowed spring 1205 may be initially bowed upward toward button 1201 and may be placed into actuator frame 1207 part way along the length of rod 1209. If button 1201 is pressed, it may compress power spring 1203 against bowed spring 1205 while a cut-out in bowed spring 1205 may engage a slot cut into rod 1209 to prevent the head of rod 1209 from moving forward. In an alternative embodiment, an outside ridge may be employed instead of a slot on rod 1209.

At a predetermined force, bowed spring 1205 may exhibit an “oil can” effect and its bow may immediately reverse orientation. This action releases rod 1209 from the ridge cut into bowed spring 1205 and rod 1209 may then be driven forward by the force built up in power spring 1203 which may then strike a sensor (not shown) with a high speed motive force for insertion.

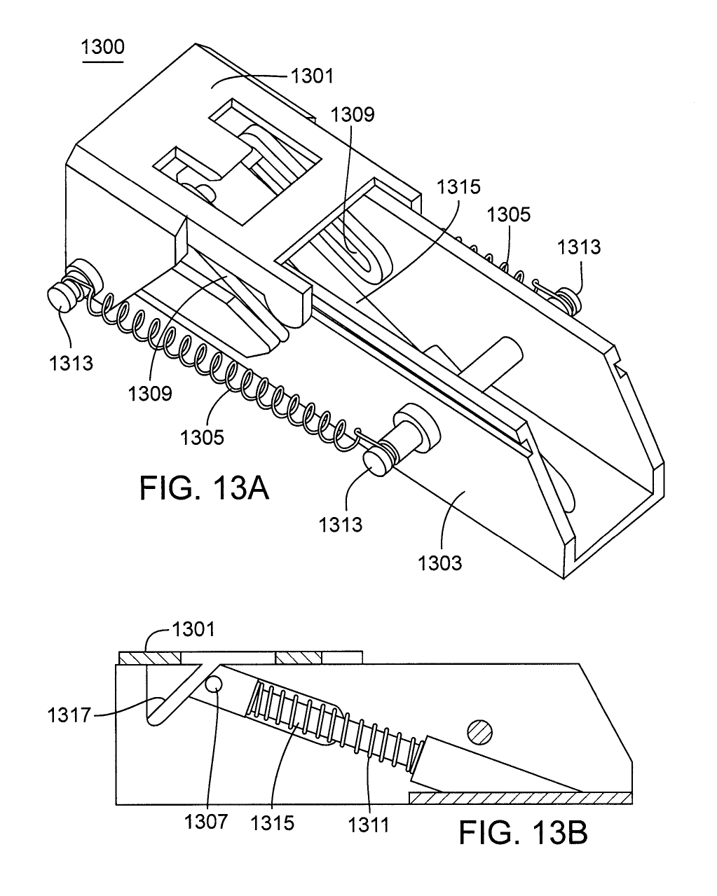

FIG. 13A depicts a mechanical spring in accordance with embodiments of the present invention. Slider 1301 may be pulled back to the far end of support structure 1303 creating tension in springs 1305 which are supported by pins 1313. Referring now to FIG. 13B which shows a cross-sectional view of the mechanical spring actuator, it may be seen that slider 1301 has an angled feature 1317 which rests against an angled surface at the top of rod 1315. Slider 1301 may be held in place by a triggering mechanism (not shown). Rod 1315 may be attached to pin 1307 each end of which sits inside two angled slots 1309 (shown in FIG. 13A) of support structure 1303. When the trigger releases slider 1301, the slider may move forward forcing rod 1315 to move in a path parallel to slots 1309 due to pin 1307. Rod 1315 may then impact a sensor (not shown) supplying a high speed motive force for insertion. Toward the end of the travel of rod 1315 its angled top feature may slip off of the corresponding angled feature of slider 1301 allowing the rod to return to its rest position using the force provided by return spring 1311. When slider 1301 is pulled back again, it may ride along a cam surface (not shown) that directs it up out of the way of the upper end of the rod and then back down behind it again, ready for the next firing.

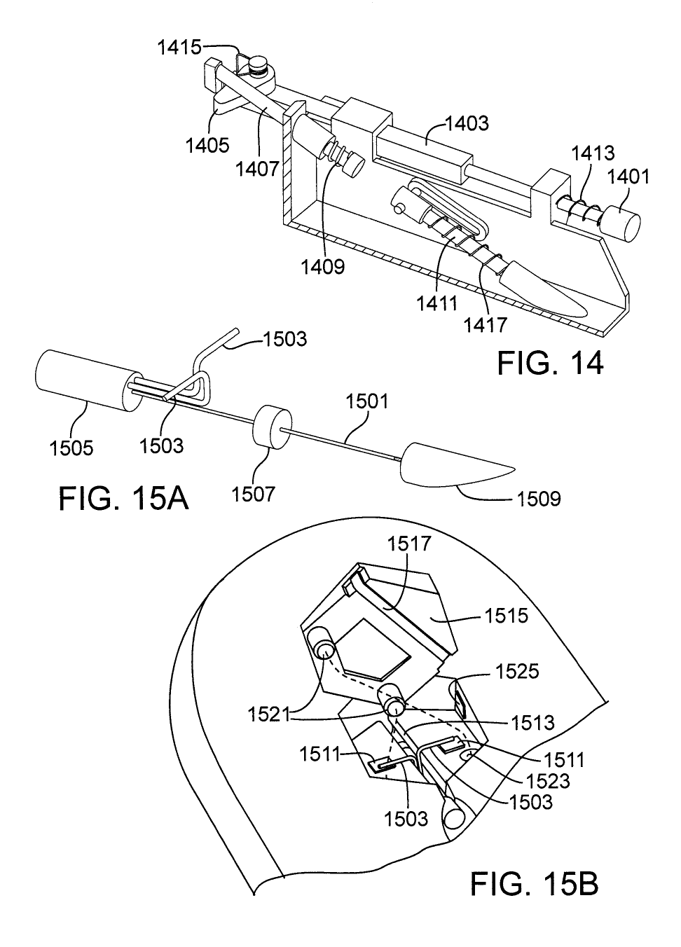

FIG. 14 depicts a cross-sectional view of a mechanical spring impact device employed to provide a high speed motive force to a sensor for insertion according to an embodiment of the invention. When button 1401 is pressed, trigger arm 1403 may be driven forward. A small shear member 1405 at the opposite end of trigger arm 1403 may be initially engaged with the top end of firing pin 1407 pulling firing pin 1407 away from rod 1411 and causing firing spring 1409 to compress and build up stored energy. As the shear moves toward the end of its travel, firing pin 1407 may slip off of the shear due to the difference in the angle of their respective travel directions. At this point, firing pin 1407 may travel forward with force supplied by compressed firing spring 1409 impacting rod 1411 and allowing the rod to impact a sensor (not shown) and supply a high speed motive force for insertion.

Subsequently, trigger arm 1403 may proceed back toward its rest position with force supplied by return spring 1413. Also, rod 1411 may proceed back to its rest position with force supplied by return spring 1417. As the shear member passes over the top end of firing pin 1407, the shear rotates to clear the upper end of firing pin 1407 and spring 1415 rotates the shear back into place to ready it for the next insertion.

FIG. 15A depicts a wiring scheme in accordance with an embodiment of the present invention. Sensor 1501 is shown with plastic bottom guide 1509 and plastic center guide 1507. Lead wires 1503 may be, in an embodiment, soldered to sensor 1501 and then insert-molded into top guide 1505. Referring now to FIG. 15B, the opposite ends of lead wires 1503 may be soldered to contacts 1511 on the body of the device. An open groove 1513 in the guidance structure may permit unobstructed movement of lead wires 1503 during sensor insertion.

Prior to insertion, pad 1515 may be partially attached to the device by partially placing pins 1521 into receptacles 1523. Upon insertion of the sensor, pins 1521 may be fully depressed into receptacles 1523 which may cause shorting bar 1517 to contact battery pads 1525 (only one shown) as pad 1515 is pushed into its final position. In this manner, shorting bar 1517 may serve to complete the power circuit of the device and turn it on.

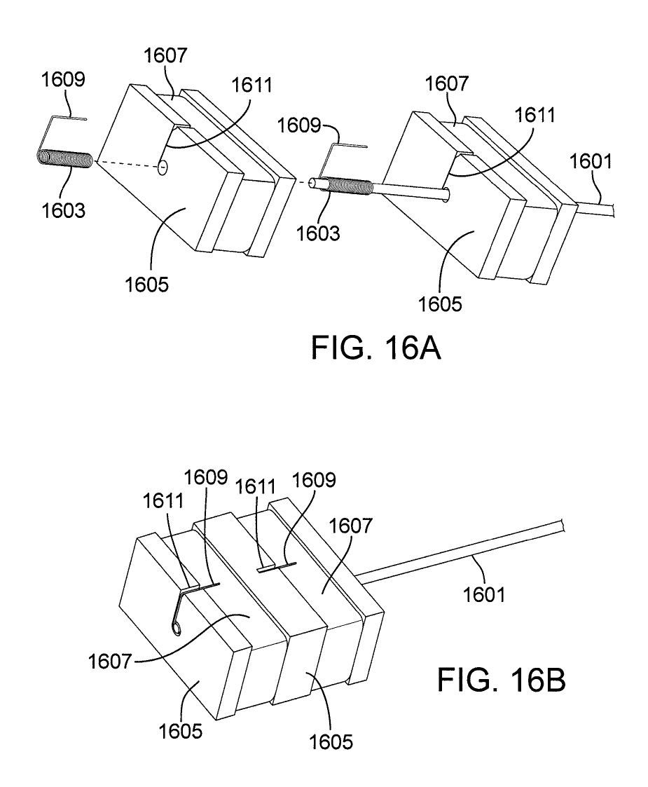

FIGS. 16A and 16B depict a sensor electrical termination assembly in accordance with an embodiment of the present invention. FIG. 16A depicts an exploded view of the embodiment. Sensor 1601 may be fitted with a set of canted coil springs 1603 positioned over the upper conductive regions of sensor 1601. Two small rectangular housings 1605 may be positioned over the springs and two rectangular sections of sheet metal 1607 may be placed into the corresponding grooves on rectangular housings 1605. Referring now to FIG. 16B, two leads 1609 extending from canted coil springs 1603 may be fed through slots 1611 in rectangular housings 1605 and spot welded onto the two sections of sheet metal 1607. Upon insertion of the sensor, this termination assembly may be moved down the insertion channel (not shown). At the bottom of the insertion channel, rectangular sheet metal 1607 may make contact with two formed spring members protruding from the channel (not shown).

An alternative approach might be to reverse the orientation of the lower of the two canted coil springs so that their leads come out of the lower end of the spring. That way, the assembly may be insert-molded into the rectangular housings to form a sealed connection.

Another embodiment includes pre-positioning the termination assembly at the bottom of the insertion channel. In that embodiment, a sensor may travel through the assembly and make electrical contact with the springs upon insertion.

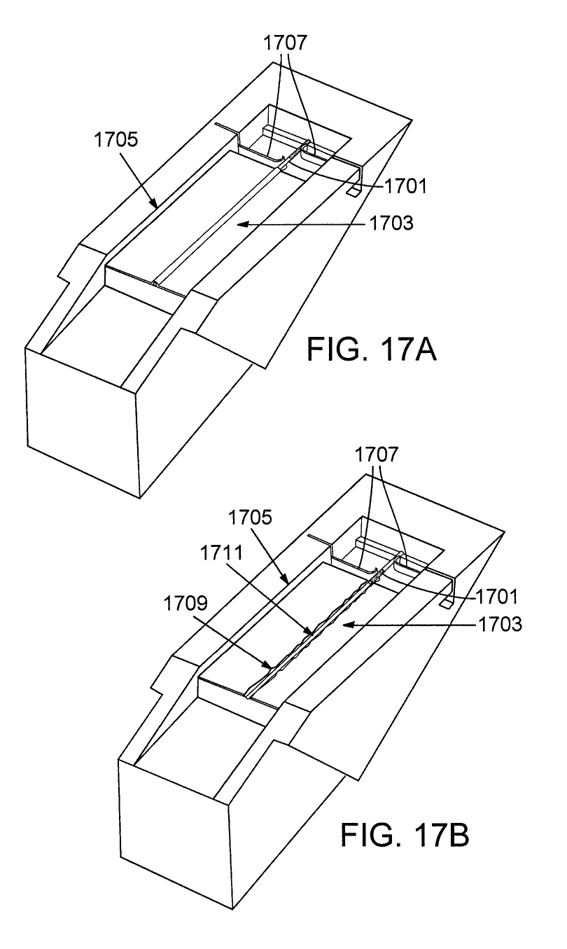

FIGS. 17A and 17B show a paper guidance structure in accordance with an embodiment of the present invention. As shown in FIG. 17A, paper 1703 may be placed inside rectangular slot 1705 and above sensor 1701. Paper 1703 may be used to secure paper 1703 prior to insertion and to guide sensor 1701 during insertion. Prior to insertion, sensor 1701 may sit inside groove 1711 (visible in FIG. 17B) at a depth of, for example, half the diameter of sensor 1701.

Referring now to FIG. 17B, an injection activation device (not shown) may push against the upper end of sensor 1701 and move inside rectangular slot 1705 during insertion. As it moves, the injection activation device may separate paper 1703 along slot 1711 creating paper tear 1709 as sensor 1701 is inserted. Upon insertion, the conductive regions of sensor 1701 may come into contact with leaf springs 1707 electrically coupling sensor 1701 to the device.

In alternative embodiments, other similar materials may be substituted for paper such as, for example, a thin plastic covering.

In an embodiment of the present invention, additional components may be housed in one or more separate modules that may be coupled to (for example, snapped to, wired to, or in wireless communication with) the insertion device. For example, the separate module may contain a memory component, a battery component, a transmitter, a receiver, a transceiver, a processor, and/or a display component, etc.

In an embodiment of the present invention, a sensor with substantially uniform cross-section may be utilized. Alternatively, in an embodiment of the present invention, a sensor with a varied cross section may be used. In embodiments, a sensor may be cylindrical, squared, rectangular, etc. In an embodiment, a sensor may be a wire-type sensor. In an embodiment, a sensor may be flexible.

Although certain embodiments have been illustrated and described herein for purposes of description of the preferred embodiment, it will be appreciated by those of ordinary skill in the art that a wide variety of alternate and/or equivalent embodiments or implementations calculated to achieve the same purposes may be substituted for the embodiments shown and described without departing from the scope of the present invention. Those with skill in the art will readily appreciate that embodiments in accordance with the present invention may be implemented in a very wide variety of ways. This application is intended to cover any adaptations or variations of the embodiments discussed herein. Therefore, it is manifestly intended that embodiments in accordance with the present invention be limited only by the claims and the equivalents thereof.

What is claimed is:

1. An insertion device comprising:
a guidance structure adapted to provide axial support to an analyte sensor, the guidance structure having an exit port; and
an injection activation device associated with the guidance structure, said injection activation device having:
a mechanism adapted to apply a high speed motive force to the analyte sensor such that, when such force is applied, the sensor moves at least partially through the guidance structure and at least partially passes through the exit port.

2. The insertion device of claim 1 wherein the mechanism adapted to apply a high speed motive force includes a device selected from the group consisting of a solenoid, a spring, a CO₂ cartridge, an air pump, and a structure adapted to maintain a sensor in a bowed configuration such that the sensor holds potential energy.

3. The insertion device of claim 1 wherein the guidance structure further comprises at least one guide member associated with the guidance structure, said at least one guide member adapted to fit inside the guidance structure and adapted to allow an analyte sensor to pass through the at least one guide member.

4. The insertion device of claim 3 wherein the at least one guide member is selected from the group consisting of a sabot, a spiral of plastic, a rectangular metallic guide, an open cell foam plastic cylinder, and a thin plastic disk.

5. The insertion device of claim 1 further comprising a housing having:
the injection activation device, said injection activation device being at least partially fixed within the housing; and
an opening aligned with the guidance structure such that a sensor initially contained entirely within the housing is able to pass through both the opening of the housing and the guidance structure upon application of high speed motive force.

6. The insertion device of claim 5 wherein the opening of the housing is flush against the exit port of the guidance structure.

7. The insertion device of claim 5 wherein the housing further comprises:
a bottom surface associated with the guidance structure, said guidance structure situated at an angle from 10 to 40 degrees with respect to the bottom surface of the housing.

8. The insertion device of claim 1 wherein the guidance structure is a tube with a circular diameter.

9. The insertion device of claim 1 further comprising an analyte sensor associated with both the injection activation device and the guidance structure and positioned such that the sensor passes through the guidance structure upon application of high speed motive force to the sensor, said high speed motive force applied by the injection activation device.

10. The insertion device of claim 9 wherein the insertion device further comprises at least one guide member associated with both the guidance structure and the analyte sensor, said at least one guide member adapted to fit inside the guidance structure, said at least one guide member adapted to allow the sensor to pass through the at least one guide member.

11. The insertion device of claim 10 wherein the at least one guide member is selected from the group consisting of a sabot, a spiral of plastic, a rectangular metallic guide, an open cell foam plastic cylinder, and a thin plastic disk.

12. The insertion device of claim 9 further comprising an electrical network coupled to the analyte sensor.

13. The insertion device of claim 9 wherein the analyte sensor is a flexible analyte sensor.

14. The insertion device of claim 1 wherein the guidance structure is a curved guidance structure.

15. The insertion device of claim 14 wherein the curved guidance structure is a curved hollow tube with a circular cross-section.

16. The insertion device of claim 14 wherein the curved guidance structure includes:
a top surface that lies at least partially outside the radius of the arc formed by the sensor during insertion; and
a partially open region that lies at least partially inside the radius of the arc formed by the sensor during insertion.

17. A method for autoinsertion of an analyte sensor into animal skin comprising:
placing an insertion device in proximal relation to animal skin, said insertion device comprising:
a guidance structure adapted to provide axial support to the analyte sensor, the guidance structure having an exit port; and
an injection activation device associated with the guidance structure, said injection activation device having:
a mechanism adapted to apply a high speed motive force to an analyte sensor such that, when such force is applied, the sensor moves at least partially through the guidance structure and at least partially passes through the exit port; and
activating the insertion device.

18. The method of claim 17 wherein the mechanism adapted to apply a high speed motive force includes a device selected from the group consisting of a solenoid, a spring, a CO₂ cartridge, an air pump, and a structure adapted to maintain the sensor in a bowed configuration such that the sensor holds potential energy.

19. The method of claim 17 wherein the insertion device further comprises at least one guide member associated with the guidance structure, said at least one guide member adapted to fit inside the guidance structure at some time during sensor insertion, said at least one guide member further adapted to allow an analyte sensor to pass through the at least one guide member at some point during sensor insertion.

20. The method of claim 19 wherein the at least one guide is selected from the group consisting of a sabot, a spiral of plastic, a rectangular metallic guide, an open cell foam plastic cylinder, and a thin plastic disk.

21. The method of claim 17 wherein the insertion device further comprises a housing having:
the injection activation device, said injection activation device being at least partially fixed within the housing; and
an opening aligned with the guidance structure such that a sensor initially contained entirely within the housing is able to pass through both the opening of the housing and the guidance structure upon application of high speed motive force.

22. The method of claim 21 wherein the opening of the housing is flush against the exit port of the guidance structure.

23. The method of claim 21 wherein the housing further comprises:
a bottom surface associated with the guidance structure, said guidance structure situated at an angle from 10 to 40 degrees with respect to the bottom surface of the housing.

24. The method of claim 17 wherein the guidance structure is a tube with a circular diameter.

25. The method of claim 17 wherein the insertion device further comprises an analyte sensor associated with both the injection activation device and the guidance structure and positioned such that the sensor passes through the guidance structure upon application of high speed motive force to the sensor, said high speed motive force applied by the injection activation device.

26. The method of claim 25 wherein the insertion device further comprises at least one guide member associated with both the guidance structure and the analyte sensor, said guide member adapted to fit inside the guidance structure, said guide member adapted to allow the sensor to pass through the guide member.

27. The method of claim 26 wherein the at least one guide member is selected from the group consisting of a sabot, a spiral of plastic, a rectangular metallic guide, an open cell foam plastic cylinder, and a thin plastic disk.

28. The method of claim 25 wherein the insertion device further comprises an electrical network coupled to the analyte sensor.

29. The method of claim 25 wherein the analyte sensor is a flexible analyte sensor.

30. The method of claim 17 wherein the guidance structure is a curved guidance structure.

31. The method of claim 17 wherein the curved guidance structure is a curved hollow tube with a circular cross-section.

32. The method of claim 17 wherein the curved guidance structure includes:
a top surface that lies at least partially outside the radius of the arc formed by the sensor during insertion; and
a partially open region that lies at least partially inside the radius of the arc formed by the sensor during insertion.
